(19) United States
(12) Patent Application Publication
(10) Pub. No.: US 2025/0019734 A1
(43) Pub. Date: Jan. 16, 2025

(54) PROCESS FOR OBTAINING PROTEINS FROM HEMP

(71) Applicant: GEA WESTFALIA SEPARATOR GROUP GMBH, Oelde (DE)

(72) Inventors: Detlef ULLMANN, Oelde (DE); Dominik KRIENKE, Bielefeld (DE); Klaus MANNWEILER, Oelde (DE); Steffen HRUSCHKA, Oelde (DE)

(21) Appl. No.: 18/707,997

(22) PCT Filed: Nov. 8, 2022
(86) PCT No.: PCT/EP2022/081050

§ 371 Date: May 7, 2024

(30) Foreign Application Priority Data
Nov. 8, 2021 (DE) 10 2021 128 968.8

(51) Int. Cl. C12P 21/00

(52) U.S. Cl. CPC C12P 21/00; C12P 2203/00

(57) ABSTRACT

Proteins are obtained from hemp by prewashing the hemp pressing remnants with addition of water, with suspension of the hemp pressing remnants to form an aqueous suspension having a pH value <7 and phase separation to form an aqueous low-protein contaminant phase containing oil and/or oil-accompanying products and a high-protein prewashed phase. Proteins are extracted by alkalizing the high-protein prewashed phase with resuspension to form an aqueous suspension and separating the phases to form a shell fraction and an aqueous protein fraction. Proteins are precipitated with the addition of a short-chain alcohol having fewer than four carbon atoms and with an acid to shift the pH value into the acidic range. The phases are separated into a low-protein phase and a high-protein phase.

### BACKGROUND AND SUMMARY OF THE INVENTION

Exemplary embodiments of the present invention relate to a process for obtaining proteins from hemp.

In principle, it is possible and also known to extract proteins from hemp. A major challenge is to remove sensory components during the process without irreversibly denaturing the proteins.

This object could not be realized with the previous processes for protein extraction from oil-containing seeds or their intermediate products after oil extraction.

Known soy protein extraction processes with leaching of the insoluble substances in the acid produce concentrates.

Processes involving extraction in alkaline solution with subsequent precipitation, on the other hand, produce isolates. The ethanolic-aqueous extraction of rapeseed or Burcon's filter and/or centrifugation processes, in which the proteins are first placed in salt solution, are well-known. What they have in common is that they either obtain protein concentrates in a purely aqueous process, starting with an alkaline step, or work with ethanol concentrations >10%.

Sunflower proteins are usually obtained in dry processes by selective separation of the shell fraction.

In the case of hemp, these processes are not used to produce oil-free protein products, as the protein product obtained is difficult to taste. The remaining bitter taste can be attributed to the presence of phenolic complexes. Processes for the production of hemp protein products with approx. 50-60% protein on dry substance can be achieved by the above-mentioned drying processes, in which the shells are separated by sieving or wind sifting. However, the unpleasant flavor components essentially remain in the protein fraction.

WO 2004/043157 A1 uses hemp seed as the starting material for the production of a hemp milk and heating to 80° C., which leads to partial denaturation of the proteins and thus to increased loss.

WO 2005/094603 A1 deals with protein-containing phytate-reduced foods using ultrafiltration. The phytate content is reduced in the protein-containing end product by ultrafiltration of the sugar-, protein- and phytate-containing extracts. Phytate and sugar-reduced protein remain in the retentate. Ultrafiltration is accompanied by the loss of filtered backwash water. Furthermore, membrane fouling can occur during prolonged use. An ultrafiltration process is particularly suitable for low solids content. If there is a quantitative precipitation of proteins, the risk of clogging of the membrane by filtrate is comparatively high. Blocking is the result.

WO 2006/003110 A1 is based on the solvent extraction of a fat from plants. However, such extraction damages the proteins.

WO 2019/213757 A1 uses microcapsules in one variant for the inclusion of fat and protein. Another variant converts hemp parts from the pressing process directly into an alkaline suspension, analogous to the soy protein isolate process. The sensory aspects are irrelevant here and are therefore not considered, as the protein is used exclusively for the production of oil-based microcapsules. Since the oil-regardless of whether it is the residual oil in the protein or the capsule oil—is decisive for the end product as a flavor carrier, the sensory component of the protein product is not considered.

The 2019 publication “Hemp (Cannabis sativa L.) Protein Extraction Conditions Affect Extraction Yield and Protein Quality” from the Journal of Food Science, Vol. 84, pp. 3682-3690, also deals with the alkaline extraction of proteins from a hemp press cake, but completely omits the removal of phenols of sensory concern. It is also pointed out that in the area of optimum protein yield, the largest proportion of phenols also ends up in the extracted protein. This means that this process is not suitable for producing a product suitable for human consumption.

Aqueous processes for sensory enhancement typically require large quantities of water. The extracted proteins are usually washed several times.

This results in high protein losses. The residual oil content in the raw material also prevents effective separation of the proteins from the organic matrix, or alternatively the oil hinders effective separation of the interfering substances from it.

Exemplary embodiments are directed to an effective, economical process for a usable protein fraction with functional properties. At the same time, the process according to the invention is intended to produce a non-denatured and sensorially acceptable protein concentrate with low proportions of oil-accompanying products, such as polyphenols.

A process according to the invention relates to the obtaining, in particular the purest possible isolation, of proteins from hemp.

The process comprises the following steps:
A Providing hemp pressing remnants, in particular a hemp press cake, in particular from the obtaining of hemp oil;
B Prewashing the hemp pressing remnants with the addition of water, with suspension of the hemp pressing remnants to form an aqueous suspension having a pH value <7 and phase separation to form an aqueous low-protein contaminant phase containing oil and/or oil-accompanying substances and a high-protein prewashed phase;
C Extracting proteins by alkalizing the high-protein prewashed phase with resuspension to form an aqueous suspension and separating the phases to form a shell fraction and an aqueous protein fraction;
D Precipitating proteins with the addition of a short-chain alcohol having fewer than four carbon atoms and with the addition of an acid to shift the pH value into the acidic range and adding alcohol; and
E Separating the phases, in particular by centrifugal phase separation, into a low-protein phase and a high-protein phase.

This process achieves a final protein content of over 70% in the final product.

Of particular interest here is the preservation of the gel-forming capacity during the processing of the protein product as a product of the process according to the invention into any consumer end products. In contrast to typical plant proteins, which show little gel-forming capacity, the hemp protein product has a high potential for gel formation and therefore this property is the focus alongside sensory properties and water binding.

The above-mentioned alcohol is preferably diluted or aqueous alcohol.

The typical process from the literature for the production of plant protein products such as isolates from de-oiled or highly oil-reduced intermediate products such as press cake or meal—based on alkaline extraction with subsequent precipitation—is not effective on its own. The new process is based on a 5-stage aqueous separation and subsequent optional drying.

Surprisingly, two measures have proven to be particularly effective, especially when these measures are implemented together as part of an overall process.

The first step is a prewash with a comparatively small amount of water. This separates a water phase with a greenish organic juice that tastes very unpleasantly aromatic and very intensely bitter. As the extract is aqueous and the CBD (cannabidiol) contained in the raw material is not water-soluble, only a marginal amount of CBD is transferred into the extract.

In contrast, oil, which is generally regarded as a flavor carrier, is also separated together with carbohydrates. This process stage is preferably carried out cold, i.e., preferably essentially at ambient temperature.

More than 50% of the added water is separated as extract. With other raw materials, the swelling is significantly higher and free water can only be separated if the amount of water is more than 5 times the amount of raw material.

Without this stage of prewashing, the final product is significantly more intense in taste and bitterness. Protein losses are limited to less than 30 wt. % of the dry mass of the extract. Typically, less than 5% protein is lost with this washing phase, relative to the amount of protein contained in the raw material.

The second measure is to carry out the precipitation in an aqueous ethanolic environment.

If available, several extracts are combined for this purpose. Surprisingly, it has been shown that even small amounts of 3-7 vol. %, in particular 5 vol. % EtOH, relative to the total amount of fluid, are sufficient to produce a compact protein phase that is significantly better in sensory terms than that from an ethanol-free precipitation.

Equally surprising is the absence of a hard foam layer as a light phase. This foam layer often occurs with other plants and/or other process control and always interferes with centrifugal separation if the EtOH is only added undiluted. In contrast, this compact foam layer does not occur when diluted EtOH is added, even if the same final concentration is set in the dispersion as with concentrated addition.

It is advantageous that in step B the dwell time is at least 1 hour from the start of suspension formation. This results in swelling of the solids in the water and thus intensive extraction. At the same time, the water is not absorbed during swelling to the same extent as is the case with the protein extraction of other plants. If water is added slowly or even continuously, the dwell time starts to form a suspension in parallel with the addition of water. A suspension is, in particular, a slurry having a liquid phase and undissolved solids in it.

The hemp pressing remnants provided in step A advantageously have a normal distribution over a mean diameter of the particles of 1.2-2.5 mm, preferably 1.5-2 mm, wherein the hemp pressing remnants are already partially de-oiled compared to a harvested hemp. Both the particle distribution and the previous partial de-oiling help with efficient prewashing and in particular the reduction of phenolic compounds in the hemp intermediate on the way to protein recovery.

Preferably, in step B, at least the same to five times the mass of water, preferably at least twice to four times the mass of water, in particular 2.5 to 3.5 times the mass of water, is added to the mass of hemp pressing remnant provided. This results in the reduction of oil and oil-accompanying products. Oil can be removed from the solids in the liquid state or dispersed in water, e.g., as an oil-water emulsion. At the same time, a large proportion of phenolic compounds are removed from the solids together with the oil and/or the emulsion.

The formation of the suspension in step B is preferably carried out at 15-50° C., preferably 20-35° C. As a result, the proteins are not thermally stressed and the solubility of proteins in wash water is kept low at the same time.

In addition, slow stirring is recommended to increase the efficiency of the wash. This can be done at an agitator speed of 3-7 m/s at the periphery of the agitator element of the agitator, e.g., the agitator blade.

The extraction in step B can be repeated at least once with the shell fraction formed during the extraction. The repeated extraction of proteins from the shell fraction enables a considerable gain in proteins of approx. 20% or even more. This depends on the protein extraction rate in the first stage. Further repetitions of the extraction result in significantly lower gains in protein yield. The conditions of the repetition can be selected analogous to the first extraction. The term shell fraction refers not only to shells, but also to hemp residues such as fibers or other solids that are produced as separated solids under the specified extraction conditions.

It is advantageous for efficient process control and optimum protein recovery if the extraction comprises at least the following steps:
C.i Suspending the high-protein prewashed phase and/or the shell fraction in water;
C.ii Alkalizing with the addition of a base, in particular NaOH, to a pH value between 9 and 11;
C.iii Phase separation, in particular centrifugal phase separation, into a solid-rich contaminant phase and a high-protein aqueous phase.

The extraction in step C can be carried out in particular at less than 55° C., in particular 40-50° C.

At least one of the aforementioned phase separations, in particular all phase separations, take place in a decanter, preferably in a separating decanter. In particular, a decanter with a full jacket and preferably a horizontal axis of rotation is used.

The concentration of the alcohol added in step D can be between 30-70 vol. %, preferably 45-55 vol. %.

The amount of water and the amount of alcohol added is adjusted in particular so that the concentration of alcohol in the suspension is less than 8 wt. %, preferably 3-7 wt. %, in particular 5+/−0.5 wt. %.

The addition of acid makes it possible to set a pH value of 5.0+/−0.8.

The alcohol added in step D can be ethanol and/or isopropanol (isopropyl alcohol), which is intended for use as a foodstuff. It is understood that ethanol or isopropanol should ideally be largely removed from the protein before the end product is made available.

The alkalizing in step C is carried out with sodium hydroxide solution, in particular with a concentration of 10-50%, especially 12-40 wt. %.

The acid is added in step D with the addition of hydrochloric acid, phosphoric acid and/or citric acid, wherein the acid is at least semi-concentrated. For example, a concentrated hydrochloric acid is about 37%, with 370 g HCl per kilogram of water.

The dwell time after the addition of acid can advantageously be at least 10 min, preferably at least 15 min. This enables the precipitation to be as quantitative as possible.

The extraction in step C is carried out at less than 55° C., in particular 40-50° C. The extraction should preferably be carried out warm in order to achieve a high protein yield. However, the protein should not denature.

The high-protein phase produced in step E can be washed by adding water and then separating the phases. The wash water from this step can be used again in steps B and C.

To reduce the risk of denaturation, it is recommended that the temperature of 55° C., preferably 50° C., is not exceeded during the entire process for obtaining proteins from hemp.

The hemp pressing remnants are advantageously prepared by mechanically pressing out the oil without adding an organic solvent. This also prevents denaturation.

### BRIEF DESCRIPTION OF THE DRAWING FIGURES

The invention is described in more detail below with reference to several embodiment variants. Identical components are provided with the same reference signs. The invention is not limited to the exemplary embodiments. In particular, individual design features from the exemplary embodiments can also be transferred to other exemplary embodiments not shown in accordance with the invention, wherein:

FIG. 1 shows the process sequence of the process according to the invention;

FIG. 2 shows a detailed process sequence of a second process step of the process of FIG. 1;

FIG. 3 shows a detailed process sequence of a third process step of the process of FIG. 1;

FIG. 4 shows a detailed process sequence of a fourth process step of the process of FIG. 1;

FIG. 5 shows a detailed process sequence of a fifth process step of the process of FIG. 1;

FIG. 6 shows a detailed process sequence of a final process step of the process of FIG. 1;

FIG. 7 shows a concentration diagram of proteins in the upper course of a decanter with changing pH value;

FIG. 8 shows a concentration diagram of proteins in different phases at different pH values and ethanol concentrations; and

FIG. 9 shows a representation of an intermediate stage of protein recovery with and without prior prewashing.

### DETAILED DESCRIPTION

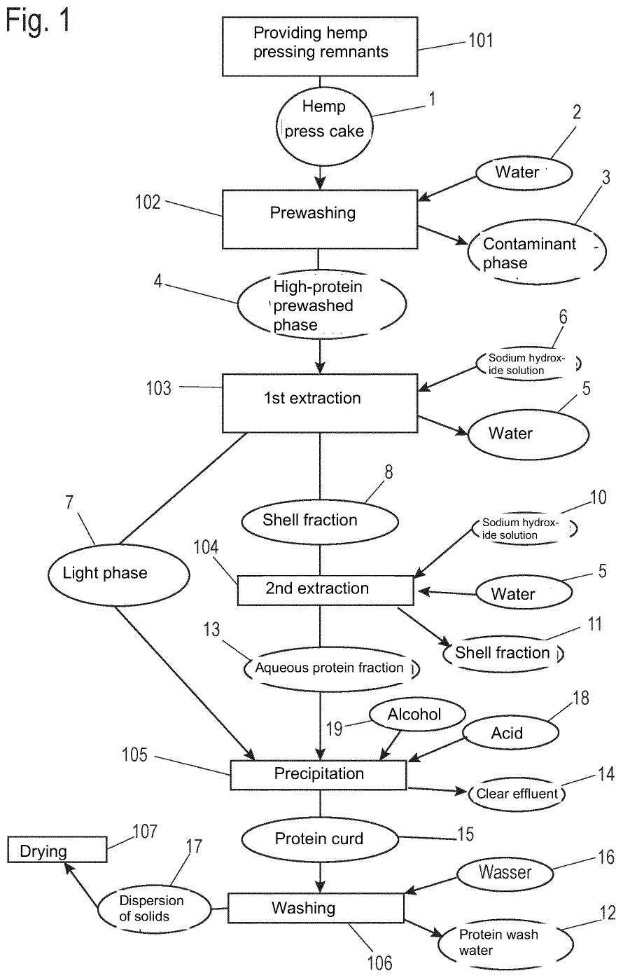

FIG. 1 shows the course of an embodiment variant of the process according to the invention.

In a first step 101, a hemp press cake 1 is provided. The hemp press cake can result from oil extraction. It itself still contains a considerable amount of residual oil.

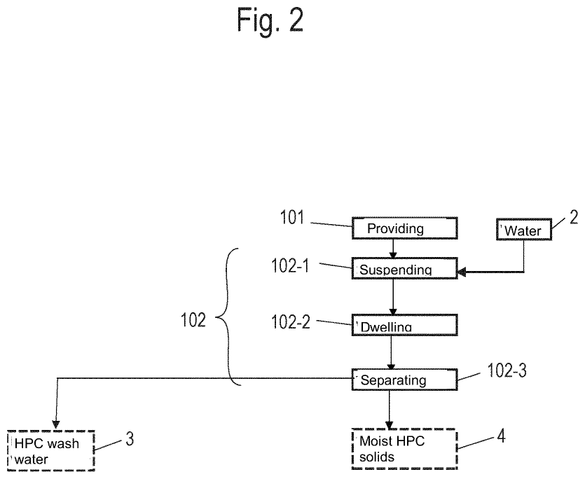

In a second step 102, a prewash is carried out, which is shown in detail in FIG. 2.

First, as part of the prewashing 102, a suspension 102-1 of comminuted hemp press cake 1 with water 2 takes place. This suspension 102-1 is also referred to below as mashing. The hemp press cake 1 preferably has a normal distribution over a mean diameter of the particles of about 1.5-2 mm.

For this purpose, three parts water are added to one part hemp press cake and mixed at 15-50° C., preferably 25° C., with an agitator speed of 3-7 m/s, preferably 5 m/s, at the periphery. This causes the solids to swell. The addition of water results in a pH value of approx. pH=6.2.

Stirring dissolves the small pieces and disperses the solids. Soluble oils and/or oil-accompanying products are dissolved in the water or specifically lighter oil is partially emulsified in the water.

Unlike other protein-containing plants, such as rapeseed, this type of prewashing is surprisingly possible with hemp press cake, as the solid matter of the hemp press cake swells to a significantly reduced extent compared to other plants.

Since oil is a flavor carrier, the taste of hemp and the products obtained from it, such as proteins, is usually very intense and bitter. However, the prewashing process makes it possible and advantageous for the oil and accompanying substances to be at least partially separated before protein extraction.

Preferably, less than six times the amount of water relative to the weight of hemp press cake is used for suspension 102-1. It has been shown that individual ingredients of a thick mash dissolve better than a thin suspension for reasons of better shearing of the solid particles.

Suspension 102-1 is followed by dwelling 102-2 as part of the prewash. This is preferably carried out with stirring. This allows the ingredients of the press cake to further dissolve or suspend in water. The dwell time 102-2 of the mash (press cake/water mixture) under stirring should preferably not be less than one hour.

This is followed by a phase separation step 102-3, preferably by centrifugation.

Since the protein does not dissolve at the aforementioned pH value, a compact protein layer is discharged together with the shells as a solid.

A separated aqueous low-protein contaminant phase 3, also known as hemp press cake wash water, contains suspended and/or dissolved oil and/or oil-accompanying products and has dry substance values of <5%, in this example 3.6% m/m. It has been observed in tests that the dry substance content of the wash water increases at higher temperatures, which means protein losses. The protein content in the wash water at 25° C. is approx. 1%, which corresponds to ⅓ of the dry substance content in the wash water. One fifth to one quarter of the dry matter in the wash water is oil. The rest is sugar, which was measured at approx. 2° Brix.

The second fraction of phase separation 102-3 is a prewashed high-protein phase 4, in particular in the form of a moist hemp press cake solids fraction. This moist washed solid with approx. 50% dry matter and 36% protein/DS and <2% oil on the dry matter is used for protein extraction in the subsequent step. A partial quantity of the wash water from the last stage can also be used as a partial quantity of the water.

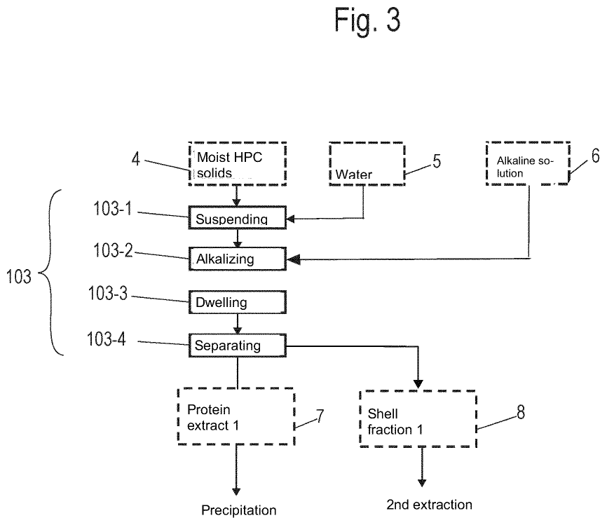

A first extraction 103 of the prewashed solid 4 then takes place, which is shown in more detail in FIG. 3. Water 5 is added to this first extraction of the prewashed solid 4. This results in renewed suspension 103-1 of the solid.

Alkalization 103-2 is then carried out by adding diluted sodium hydroxide 6. The concentration can be adjusted (concentration 10-50 wt. % based on the amount of sodium hydroxide) to a pH value of 9 to 11, preferably 10.

Enough fluid, i.e., water and lye, must be added so that the dry substance content in the suspension is approx. 20-26% m/m, preferably 23% m/m.

The product is then left to dwell for a further time 103-3, preferably at 10-55° C., particularly preferably for at least 10 minutes, especially at least 30 minutes. Dwelling can be carried out with stirring. Alternatively, a dwell time is only necessary until the working temperature for the subsequent separation step is reached. The stirrer speed is optimal for up to 3 m/s at the periphery.

Subsequent phase separation 103-4 can preferably be carried out centrifugally and particularly preferably by a decanter. This separates the suspension into approximately ⅔ light phase 7 and ⅓ heavy phase of the shell fraction 8. The working temperature during phase separation is preferably less than 60° C., preferably 40-55° C., in particular 50° C. The light phase 7 comprises an aqueous extract with a high protein content.

The shell fraction 8 is fed to a second extraction stage. The protein extract 7 is fed to a precipitation stage in step 4, together with the protein extract from the second stage or any other extracts produced from further extraction stages. This will be explained in more detail below.

A partial quantity of the wash water from the last stage of the process, which is described below as protein wash water 12, can also be used as a partial quantity of water 5.

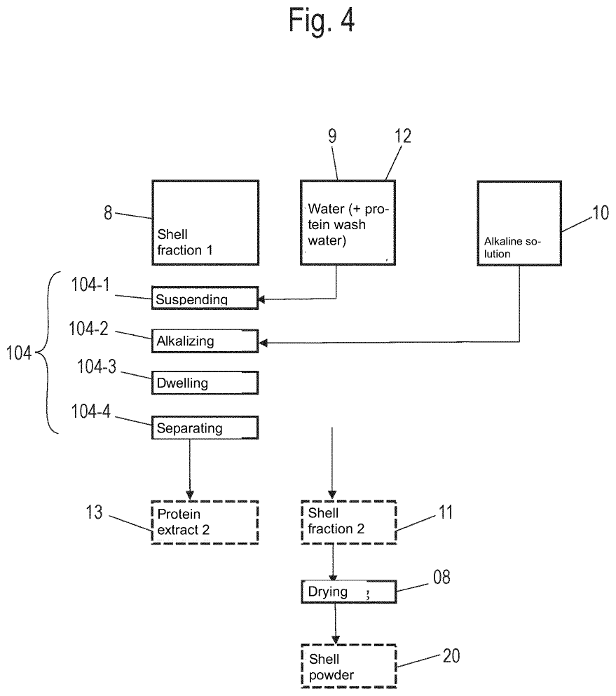

The solid 8 from the first extraction is used for the second extraction 104, as shown in FIG. 4.

This is resuspended with water 9 at a rate of approx. 70-130 wt. %, based on the solids used. The protein wash water 12 can also be partially used for this purpose. After suspension 104-1, alkalization 104-2 takes place with the addition of sodium hydroxide solution 10. The pH value should preferably be raised to at least 9.0 and particularly preferably to pH=9.6-10, since it has fallen as a result of the water dilution.

The dry substance value before separation can advantageously be approx. 22%+/−3% m/m. The working temperature can preferably be in the range of maximum 55° C., preferably 40-50° C.

The alkalization 104-2 is optionally followed by a dwelling 104-3 with stirring. The optional dwell time is preferably less than 20 min, preferably 0-15 min.

Subsequent phase separation 104-4 is preferably carried out centrifugally and in particular with a decanter. A shell fraction 11 and an aqueous protein fraction 13 are formed.

The shell fraction 11 of this process stage can preferably be further processed into shell powder 20 by drying 108, either directly or after a pH correction to a pH value in the neutral range at pH=6.5-7.5.

It is also possible to use the shell fraction 11 directly or to dispose of it. A residual protein content of 10-15% remains in the shell fraction 8 and can be reduced by further extraction stages. However, the main component of the shell powder 20 is the dietary fiber.

Extract 13 from the second extraction 104 contains approx. 5% dry matter, of which approx. ⅔ are proteins. The protein content is thus higher in percentage terms than that in extract 7 from the first extraction 103. In contrast, the oil content of <1% relative to the dry matter is lower than that in the first extract 7 at 1-2% m/m.

Both high-protein aqueous fractions 13 together contain approx. 60-70% of the protein contained in the raw material, with approx . . . 80% of this coming from the first extraction and 20% from the second extraction.

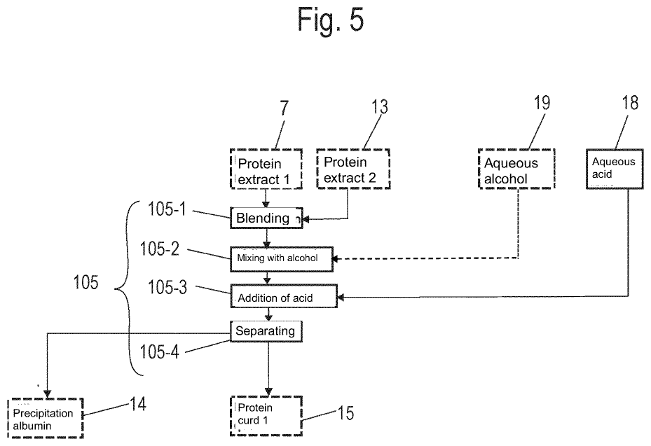

The second extraction 104 is followed by the aforementioned precipitation step 105. This is explained in more detail in FIG. 5.

In this process stage, the extracts from the previous extraction stages are mixed together by blending 105-1.

Subsequently, in a mixing step, aqueous alcohol 19, 105-2, in particular aqueous ethanol, is added in a concentration of 30-70 wt. %, preferably 50+/−5 wt. %, so that the final concentration of ethanol in the fluid of the suspension is 3-7 vol. %, preferably 5 vol. %. The addition of a higher concentration of alcohol, e.g. 90 vol. %, led to the formation of a protein flotate which is difficult to separate.

The result is a mixture with approx. 10%+/−3% dry substance of approx. 58 wt. %+/−3 wt. % protein in relation to the dry matter and less than 2 wt. % oil in the dry matter content.

The protein is then precipitated by adjusting the pH value 105-3, in particular by adjusting the isoelectric point, which has a pH value of 5.0+/−0.8. Acid 18, in particular hydrochloric acid in a concentration of 35% by mass, is used for this purpose.

Other acids such as phosphoric acid or citric acid in concentrations of up to 50% by mass could also be used additionally or alternatively. Precipitation takes place at temperatures of 40-55° C., preferably at 50° C.

Optionally, a dwell time can be added. The dwell time is not necessary in any process step in precipitation stage 105, but a dwell time of at least 10 min after addition of the acid is advantageous for flocculation of the protein. Stirring should also be carried out during the process steps, at max. 3 m/s at the periphery of the stirrer.

The addition of acid 105-3 produces two phases, a clear phase 14 and a heavy phase (protein curd) 15, which are separated from one another by phase separation, in particular centrifugal phase separation 105-4, especially preferably by a decanter, in particular a separating decanter. The clear phase 14 usually has a dry substance content of approx. 2%, wherein this also contains oil, with approx. 10% based on the dry mass in this clear phase. This means that the protein curd with approx. 20%+/−4% dry substance is significantly lower in oil, namely only 0.6%+/−0.4%.

Furthermore, the majority of the substances that are soluble in the ethanolic-aqueous environment go into the clear phase. This is due to the fact that the fluid content in the precipitated albumin corresponds to more than 60% of the fluid in the suspension, which is separated in this precipitation stage. Some of the dissolved substances are aroma components (phenolic complexes).

The proportion of total protein that is separated as albumin with the light phase is significantly smaller than the proportion of protein that is separated as globulin with the protein curd. Typical ratios here are 5 to 8 wt. % albumin protein in relation to the amount of protein in the globulin.

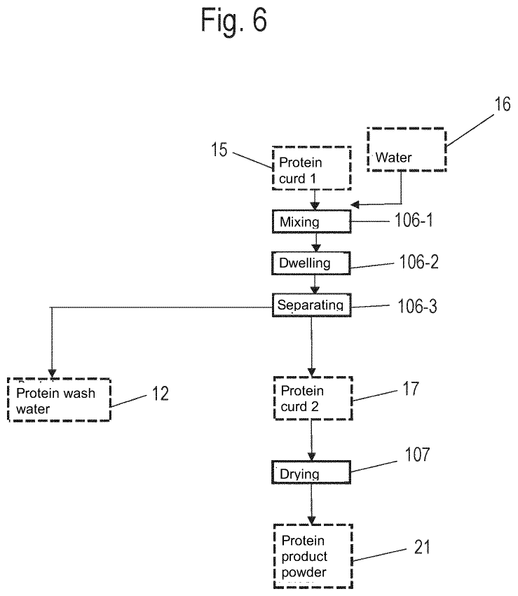

The final stage of the process is the washing 106 of the protein 15. This is shown in FIG. 6. Here, water 16 is added to the protein curd 15 from the precipitation stage 105 in the preferred proportion of 70-130 wt. %, a suspension is formed by mixing 106-1, a favorable washing out of soluble constituents is achieved by dwelling 106-2 and then separated again in the separation step 106-3.

The mixture thus has a dry substance value of approx. 10% m/m. In terms of volume, approx. 50% is obtained as washed curd with approx. 20% dry substance m/m as a solid suspension (curd).

Mixing takes place in the stirred tank at stirring speeds of up to 3 m/s at the periphery of the stirrer. The dwell time is relatively short, at least 5 min, preferably 6-15 min.

The temperature is about 40-55° C., preferably 50° C. The separated wash water 12 contains less than 2% dry substance and can be fed to the first and/or second extraction step 103, 104 as protein wash water.

According to the invention, the separation is carried out centrifugally with a separating decanter into a curd-like solid dispersion 17 and an almost aqueous overflow as protein wash water. After purification, the wash water can optionally be used as dilution water for the first and/or second extraction.

Alternatively, it can also be used as wash water for the prewash 102. The latter even has the advantage that the wash water of the prewash is lost water and thus no or only a very low intermediate enrichment of washed-out flavoring substances and oil components in the extract takes place.

The protein losses in the wash water 12 are less than 1 wt. % in absolute terms. At less than 2 wt. % of the protein in the raw material, this represents a small amount. In contrast, the protein content in the washed protein curd remained almost unchanged at over 70%, even over 73%, compared to the value before washing.

From a sensory point of view, there is a clear reduction in the bitter and hemp-typical taste due to the washing step 106.

Finally, the high-protein protein curd 17 is dried 107, in particular below 55° C. and preferably optionally under negative pressure, to form a protein powder 21.

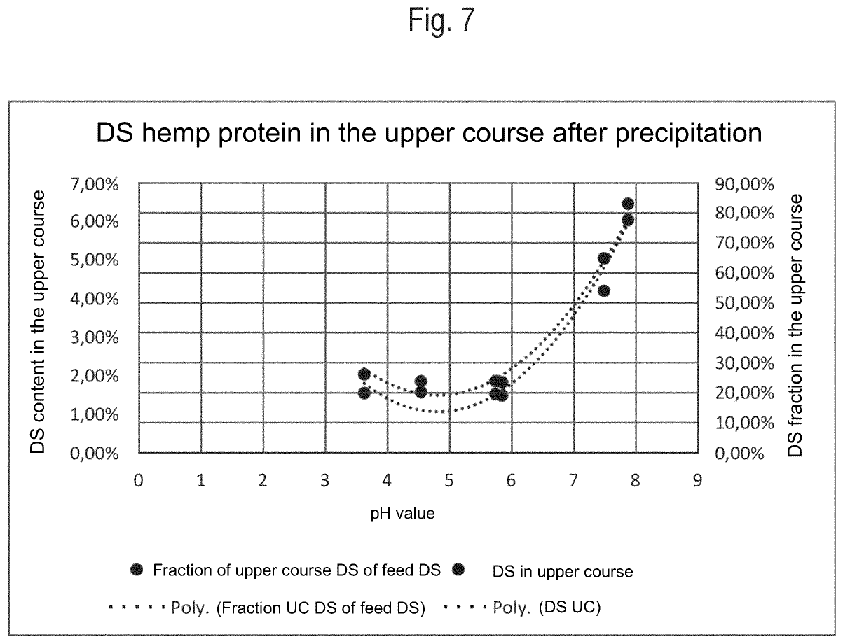

FIG. 7 again shows the influence of the pH value on the dry substance content in the upper course. This overflow is the clear phase after precipitation. To keep the loss of protein relatively low, the protein content of this phase should be kept as low as possible.

As can be seen, the transition of proteins into the clear phase is ideal in the isoelectric points described above, so that no product losses occur.

Encapsulation of the proteins, which would require additional separation of the capsule material, does not take place in the process. In addition, the temperature in the process is not increased to over 55° C., which would promote denaturation.

Likewise, no additional salt is added in quantitative quantities for the purpose of salting out during the entire process of extracting proteins from hemp.

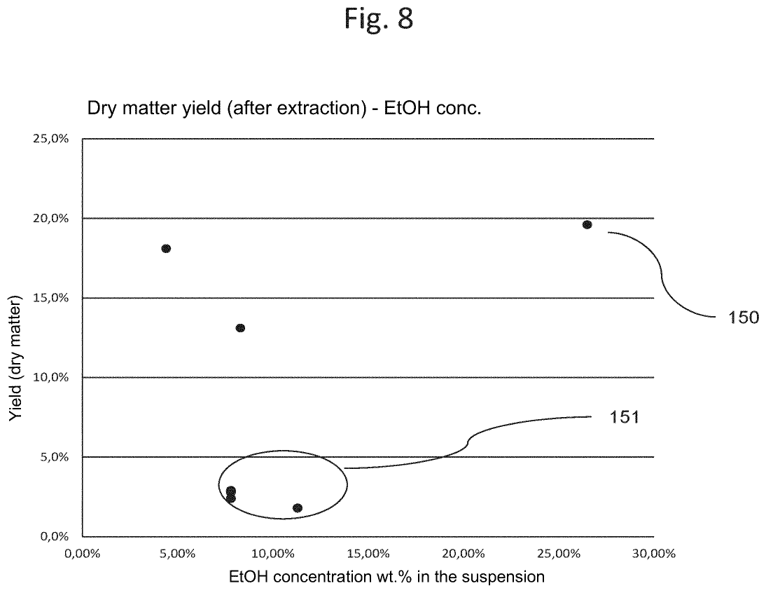

FIG. 8 shows a diagram of the dry matter yield in relation to the ethanol concentration after precipitation in step D at neutral to acidic pH.

The marked measuring points 151 show the proportion of dry matter yield in the alcoholic-aqueous phase after the addition of ethanol at neutral pH value and the upper measuring points 150 show the proportion of dry matter in the separated protein phase after the addition of acid.

It can be seen that regardless of the ethanol concentration, little protein-containing dry matter is produced in the ethanolic-aqueous phase. It was concluded from this that prewashing with water at a neutral pH value leads to very low product losses.

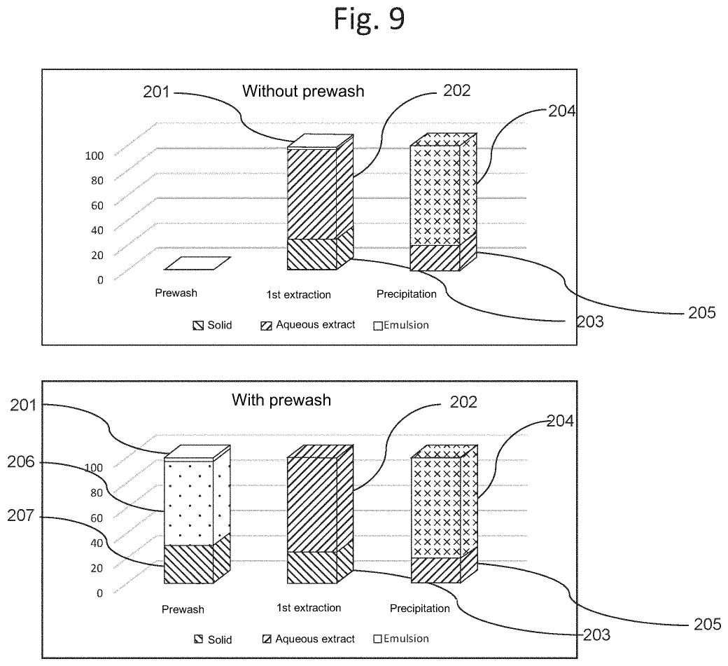

FIG. 9 schematically shows the phases of centrifuge tubes after centrifugal treatment at the respective stages of the process. A first illustration shows a process sequence with prewashing and a second illustration shows a process sequence without prewashing.

For the process without prewashing, an emulsion phase 201 comprising oil, proteins, and possibly water is obtained after the first extraction.

Furthermore, an aqueous extraction phase 202 containing proteins, sugars and undesirable phenols as accompanying oil substances is obtained.

In addition, a solid phase 203 consisting of shells, protein, phenols, and other compounds is obtained.

After the pH shift and precipitation in step D, the following phases are then obtained. A liquid extract phase 204 comprising dissolved substances such as phenols and a solid phase 205 comprising proteins.

Similar observations were made during the process run with the prewash, however, an emulsion 201 comprising oil and proteins was already formed during the prewash. Furthermore, a liquid phase 206 was formed comprising sugar, phenol and less than 5 wt. % proteins.

In addition, a solid phase 207 was formed which comprises shells, proteins, phenols, and the like.

Due to the comparatively pure phases 206 and 207 after the prewash, a liquid phase 202 comprising proteins, sugars and phenols is formed after extraction.

The solid phase 203 itself only contained shells and proteins. Finally, precipitation takes place, providing a protein phase 205 and a liquid phase 204.

The phases are divided up as follows:

For the first extraction (without prewash): 25 vol. % solids 203, 72 vol. % liquid phase 202 and 2 vol. % emulsion 201.

For precipitation (without prewash): 20 vol. %. Solids (proteins) 205 and 80 vol. % aqueous-ethanolic phase 204.

For the prewash: 30 vol % solids 207, 67 vol % aqueous extract 206 and 3 vol. % emulsion 201

For the first extraction (with prewash): 25 vol % solids 203, 75 vol % liquid phase 202.

For precipitation (without prewash): 20 wt. %. solids (proteins) (205) and 80% aqueous-ethanolic phase (204).

The proportion of polyphenols in the protein phase in the variant without prewashing is higher than in the variant with prewashing.

A special feature of obtaining protein from hemp is the formation of a gel in the high-protein phase. This gel has a higher viscoelasticity compared to water and the other fractions. The gel is therefore a fluid with a higher viscoelasticity compared to water and the other fractions occurring in the process. This is also surprising in that this rheological peculiarity does not occur in other plants, e.g., rapeseed, or at least not to this extent.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### LIST OF REFERENCE SIGNS

1 Hemp press cake
2 Water
3 Aqueous low-protein contaminant phase
4 High-protein prewashed phase
5 Water
6 Diluted sodium hydroxide solution
7 Light phase
8 Shell fraction
9 Water
10 Diluted sodium hydroxide solution
11 Shell fraction
12 Protein wash water
13 Aqueous protein fraction
14 Clear phase
15 Protein curd
16 Water
17 Solid dispersion
18 Acid
19 Alcohol
20 Shell powder
21 Protein powder
101 Provision of hemp pressing remnants
102 Prewash
102-1 Suspension
102-2 Dwelling
102-3 Phase separation
103 Extraction
103-1 Suspension
103-2 Alkalization
103-3 Dwelling
103-4 Phase separation
104 Second extraction
104-1 Suspension
104-2 Alkalization
104-3 Stirring
104-4 Phase separation
105 Precipitation
105-1 Blending
105-2 Addition of aqueous alcohol
105-3 Adjusting the pH value
105-4 Phase separation
106 Washing
106-1 Suspension
106-2 Dwelling
106-3 Separation
107 Drying
108 Drying

1-23. (canceled)

24. A method for obtaining proteins from hemp, the method comprising:
A) providing hemp pressing remnants;
B) prewashing the hemp pressing remnants with addition of water, with suspension of the hemp pressing remnants to form an aqueous suspension having a pH value <7 and phase separation to form an aqueous low-protein contaminant phase containing oil or oil-accompanying products and a high-protein prewashed phase;
C) extracting proteins by alkalizing the high-protein prewashed phase with resuspension to form an aqueous suspension and separating phases of the aqueous suspension to form a shell fraction and an aqueous protein fraction;
D) precipitating proteins with addition of a short-chain alcohol having fewer than four carbon atoms and with addition of an acid to shift the pH value into the acidic range and produce a clear phase and a heavy phase; and
E) separating the clear and heavy phases into a low-protein phase and a high-protein phase.

25. The method of claim 24, wherein a dwelling in step B) occurs over at least 1 hour from a start of the formation of the suspension.

26. The method of claim 24, wherein the hemp pressing remnants provided in step A) have a normal distribution over a mean diameter of the particles of 1.2-2.5 mm, and wherein the hemp pressing remnants are already partially de-oiled compared to a harvested hemp.

27. The method of claim 24, wherein the hemp pressing remnants are provided by mechanical pressing of oil without addition of an organic solvent.

28. The method of claim 24, wherein in step B) at least 1-5 times a mass of water is added to a mass of pressed hemp residues provided.

29. The method of claim 24, wherein the formation of the aqueous suspension in step B) occurs at 15-50° at an agitator speed of 3-7 m/s at a periphery of an agitator element of an agitator.

30. The method of claim 24, wherein the extraction in step C) is repeated at least once with the shell fraction formed during the extraction.

31. The method of claim 30, wherein the extraction or the repeated extraction comprises at least:
C.i) suspending the high-protein prewashed phase or the shell fraction in water;
C.ii) alkalizing with the addition of a base to a pH value between 9 and 11;
C.iii) phase separation into a shell fraction and a high-protein aqueous phase.

32. The method of claim 24, wherein the extraction in step C) is performed at less than 55° C.

33. The method of claim 31, wherein at least one of the aforementioned phase separations takes place in a decanter.

34. The method of claim 24, wherein a concentration of the short-chain alcohol added in step D) is between 30-70 vol. %.

35. The method of claim 34, wherein an amount of water and an amount of short-chain alcohol added is adjusted such that a concentration of the short-chain alcohol in the suspension in step D) is less than 10 wt. %.

36. The method of claim 24, wherein the acid is added in step D) to set a pH value of 5.0+/−0.8.

37. The method of claim 24, wherein the short-chain alcohol added in step D) is ethanol or isopropanol.

38. The method of claim 24, wherein the alkalizing in step C) is performed with alkali with a concentration of 10-50 wt. %.

39. The method of claim 24, wherein the addition of the acid in step D) is performed with the addition of hydrochloric acid, phosphoric acid or citric acid, wherein the acid is present in at least semi-concentrated form.

40. The method of claim 24, wherein a dwell time after the addition of acid is at least 10 min.

41. The method of claim 24, wherein the extraction in step C) is performed at less than 55° C.

42. The method of claim 24, wherein a washing is performed with the high-protein phase from step E) by adding water and subsequent phase separation.

43. The method of claim 42, wherein the water of the washing is reused in step B) or C) in a repeat step of the extraction of the shell fraction.

44. The method of claim 24, wherein a temperature of 55° C. is not exceeded during the entire method for obtaining proteins from hemp.

45. The method of claim 24, wherein no additional salt is added during the entire method for obtaining proteins from hemp.

46. The method of claim 24, wherein the high-protein phase in step E) comprises a gel or is formed as a gel.
